(19) United States
(12) Patent Application Publication
(10) Pub. No.: US 2012/0094361 A1
(43) Pub. Date: Apr. 19, 2012

(54) Method of Separation of Algal Biomass from Aqueous or Marine Culture

(75) Inventors: Qiang Hu, Chandler, AZ (US); Milton Sommerfeld, Chandler, AZ (US); Yongsheng Chen, Gilbert, AZ (US); Xuezhi Zhang, Chandler, AZ (US)

(73) Assignee: Arizona Board of Regents, a body of the State of Arizona, Acting for and of Behalf of Arizona State, Scottsdale, AZ (US)

(21) Appl. No.: 13/259,949

(22) PCT Filed: Apr. 15, 2010
(86) PCT No.: PCT/US10/31181

§ 371 Date: Dec. 19, 2011

Related U.S. Application Data
(60) Provisional application No. 61/170,470, filed on Apr. 17, 2009.
(60) Provisional application No. 61/172,293, filed on Apr. 24, 2009.

(51) Int. Cl. C12N 1/12

(52) U.S. Cl. 435/257.1

(57) ABSTRACT

Disclosed are cross-flow membrane filtration methods for the removal or separation of algal cells from an aqueous environment. The methods of the invention may be used for the simultaneous algal harvesting/dewatering and water/wastewater purification and recycling.

### RELATED APPLICATIONS

The present application is based on U.S. Provisional Patent Application No. 61/170,470, which was filed Apr. 17, 2009, and U.S. Provisional Patent Application No. 61/172,293, which was filed Apr. 24, 2009. The entire text of the aforementioned applications is incorporated herein by reference in its entirety.

### FEDERALLY SPONSORED RESEARCH OR DEVELOPMENT

[Not Applicable]

### BACKGROUND OF THE INVENTION

A major drawback of the currently available methods of biomass cultivation for biofuel production is the lack of an economical and efficient method to harvest biomass (1). Biomass harvesting is a challenge because of the small size (3-30 μm diameters) of the algal cells, their similar density to water, and the large water volumes that must be handled to recover the biomass. (The collection of 10 kg of algal biomass from a 3 g/L algae suspension requires 3,300 L of water.) Recovery of the biomass from the culture medium may contribute 20 to 30% of the total cost of producing the algal biomass (2).

Algae harvesting requires one or more solid-liquid separation steps, including the concentration and drying processes. The most frequently used concentration technologies are coagulation, flocculation, flotation, centrifugation, filtration (screen, membrane) and gravity sedimentation (3-9). Among these technologies, membrane technology is very promising. Membrane filtration can remove bacteria from used algal culture media, which impact algae growth, while retaining residual nutrients; thus, the algal culture media can be recycled. A polyacrylonitrile ultrafiltration membrane with a 40-kDa molecular weight cutoff was found to be satisfactory for the continuous recovery of two marine microalgae (Haslea ostrearia and Skeletonema costatum) (10). Petrusevski et al. examined a tangential flow filtration system for the concentration of living freshwater phytoplankton from large volumes of reservoir water with low algal biomass. Samples were concentrated 5 to 40 times using a 0.45 um pore-size membrane (11).

However, the fouling of the membrane is a major problem in biomass concentration and the currently available methods do not yield an effective concentration of biomass on an industrial scale (12-16). To develop an efficient strategy for biomass concentration using membranes, it is necessary to obtain a detailed characterization of the foulants and to develop useful anti-fouling strategies that can be used to provide appropriate dewatering of biomass without the production costs and inefficiencies of the currently available systems.

### BRIEF SUMMARY OF THE INVENTION

The invention provides a cross-flow membrane system and method for the removal or separation of algae from an aqueous environment or medium. Once removed the algae may subsequently be processed by any methods typically employing algae for biofuel and/or bioproduct production. The methods of the invention also advantageously purify the water or the medium in which the algae are contained so that the medium or water may be re-used or further processed. Thus, the methods of the invention provide the dual advantages of harvesting and dewatering of algal biomass and purification of water/wastewater or media for further use or recycling.

In specific exemplary embodiments, the present invention achieves the above-described advantages by dewatering an aqueous algal biomass comprising using the steps of subjecting an initial aqueous algal biomass to a continuous cross-flow ultrafiltration to remove water from the aqueous algal biomass, wherein the ultrafiltration is performed using a hollow-fiber PVC ultrafiltration membrane with a molecular weight cutoff off of 50 kDa; and intermittently subjecting the ultrafiltration membrane to an air-based backwash to remove algae from the membrane wherein the method produces at least a 50-fold increase in algal biomass in the aqueous suspension as compared to the algal biomass in the starting aqueous suspension.

In certain embodiments, the method further comprises collecting the aqueous permeate removed from the algal biomass.

In exemplary embodiments, the initial aqueous biomass comprises an algal biomass of 1.04 g/L cell dry weight, and the continuous ultrafiltration produces an aqueous algal biomass suspension comprising about 50 to 100 g/L cell dry weight.

In certain exemplary methods, the method further comprises monitoring the average flow rate of the permeate through the membrane.

In specific embodiments, the air-based backwash is performed at a time interval selected from the group consisting of every 10 minutes, every 15 minutes, every 20 minutes, every 25 minutes, every 30 minutes, every 35 minutes, every 40 minutes, every 45 minutes, every 50 minutes, every 55 minutes and every 60 minutes. However, it should be understood that the backwash interval depends on how fast the flux decline is. It can be also set as 26, 27, even 27.5 min. As such the backwash may be set at any desirable minute interval between 10 minutes and 60 minutes.

In other methods, the method further comprises declogging the membrane to remove algogenic organic matter thereform.

In exemplary methods the membrane may be declogged by air-based backwash when the flux of the permeate through the membrane decreases to a value between 20 to 70 L/m²h. These are exemplary data based on use of PVC membranes such as those disclosed in U.S. Pat. No. 7,435,348 (incorporated herein by reference). It should be understood however that the collection method described herein also may be performed with other membranes such as PVDF, PE, CA membranes which may have varying but nevertheless effective flux for use in declogging.

The declogging of the membrane may be performed when the flow rate of the permeate through the membrane decreases to less than 50% of the flow rate of the permeate through new membrane.

Alternatively, the membrane is declogged when the average flow rate of the permeate through the membrane decreases to less than 50% of the average flow rate of the permeate through a membrane immediately after chemical declogging.

In yet another alternative embodiment, the membrane is declogged when the flux of the permeate after backwashing dropped to less than 60% of the initial flux of the permeate through the membrane.

In specific embodiments, the declogging comprises a soaking step wherein the cross-flow ultrafiltration is stopped and the ultrafiltration membrane is soaked with a circulating fluid comprising cleaning solutions selected from the group consisting of about 1% to about 5% NaOH, about 0.1% to about 2% citric acid, about 50 mg/L to about 1000 mg/L NaCIO, about 0.5% to about 5% EDTA and about 0.5% to about 5% surfactants.

In preferred embodiments, the cleaning solution comprises about 400 mg/L NaClO.

In exemplary embodiments, the aqueous algal biomass is contained in a freshwater sample. In other embodiments the algal biomass is contained in a marine water sample. In still other embodiments, the algal biomass is contained in a growth medium for algae. In still other embodiments, the algal biomass is contained in a waste-water sample.

The algal biomass may contain any algae or microalgae including one or more algae belonging to a family of selected from the group consisting of Chlorophyceae, Prasinophyceae, Bacillariophyceae, Cryptophyceae, Chrysophyceae, Haptophyceae and Cyanophyceae.

In specific embodiments, the starting aqueous suspension comprises between 1 to 100×10⁶ algal cells/mL. In preferred embodiments, the method is preformed to concentrate such a starting aqueous suspension such that for example, the method concentrates the algal cells in the suspension to a concentration of between about 1×10⁶ to 30×10¹⁶ cells/mL.

Also contemplated herein is an isolated aqueous suspension of algae isolated according to the method of the invention wherein at least 90% of the algal cells in the population are live, unruptured algal cells.

The invention further provides a method of recycling an aqueous solution containing an algal biomass comprising the steps of: subjecting an initial aqueous solution containing algae and other microorganisms to a continuous cross-flow ultrafiltration to remove water from the aqueous algal biomass, wherein the ultrafiltration is performed using a hollow-fiber PVC ultrafiltration membrane with a molecular weight cutoff off of 50 kDa; intermittently subjecting the ultrafiltration membrane to an air-based backwash to remove algae from the membrane; and collecting the permeate isolated from the ultrafiltration step.

The method may further comprise declogging the membrane to remove some of the algogenic organic matter therefrom. In specific embodiments, the aqueous solution is an algal culture growth medium. In other embodiments, the aqueous solution is a waste water sample, a marine water sample or a fresh water sample containing algae.

Specific embodiments provide a cross-flow membrane filtration method for the removal or separation of algal cells from an aqueous environment by subjecting an initial aqueous algal biomass to a continuous cross-flow ultrafiltration to remove water from said aqueous algal biomass, wherein said ultrafiltration is performed using a hollow-fiber PVC ultrafiltration membrane with a molecular weight cutoff off of 50 kDa; subjecting said ultrafiltration membrane to an air-based backwash to remove algae from said membrane at least once every 30 minutes; and chemically declogging said ultrafiltration membrane when the average flow rate of the permeate after backwashing dropped to less than 60% of the initial average flow rate of the permeate through the membrane; wherein said method produces at least a 50-fold increase in algal biomass in said aqueous suspension as compared to the algal biomass in the starting aqueous suspension.

### BRIEF DESCRIPTION OF SEVERAL VIEWS OF THE DRAWINGS

FIG. 1: Schematic of an apparatus used in the methods of the present invention.

FIG. 2: Schematic of the model calculation process. i is number of the filtration cycle, and j is number of the calculated step at each cycle (j=1, 2, 3 . . . min).

FIG. 3: Analysis of the flux decline due to different foulants.

FIG. 4A and FIG. 4B: Scanning electron microscope (SEM) images of the virgin membrane (A) and fouled membrane (B).

FIG. 5: Development of algal cake thickness and algal concentration as a function of time.

FIG. 6: Fourier transformed infrared (FTIR) spectra of FTIR spectra of the fouled membrane (a) and virgin membrane (b).

FIG. 7: Effect of cross flow velocity on flux decline as a function of time.

FIG. 8: Effect of algal concentration on flux decline as a function of time.

FIG. 9: Flux decline during the long term concentration experiment.

FIG. 10: Volumetric reduction factor (VRF) profile during the algae concentration process.

### DETAILED DESCRIPTION OF THE INVENTION

Concentration of biomass using membrane technology is very promising, as the water obtained can be reused. However, during this process the fouling of the filtration membrane due to the increase in the algae concentration is a rate-limiting problem and filtration methods have not proven effective. To develop efficient biomass concentration strategies using the membrane technique, the present invention characterizes the foulants using SEM and FTIR, and optimizes the operation parameters. A model was also developed to describe the flux decline based on a resistance-in-series analysis and a cake development calculation.

The results shown herein below teach that during filtration methods a buildup of the algal cake layer and adsorption of algogenic organic matter (AOM) (mainly protein, polysaccharides or polysaccharides) on the membrane cause backwash reversible and backwash irreversible fouling of the membrane. The cake layer buildup can be removed by conducting an air assisted backwash every 15 min. The adsorbed AOM can be removed by soaking the membrane using 400 mg/L NaClO for 1 hour. Under optimized operating conditions, the harvesting efficiency and average permeability were 46.01 g/m²h and 45.50 L/m²h, respectively. No algae were found in the permeate, which had an average turbidity of 0.018 NTU. The flux decline predicted by the model at different conditions was consistent with the experimental results.

In the methods described herein, the aqueous algal biomass suspension prior to being concentrated can have a concentration ranging from 1×10⁶ to 500×10⁶ cells/mL and preferably from 1×10⁶ to 50×10⁶ cells/mL. Typically, the suspension prior to being concentrated according to the methods described herein is a fresh culture of microalgae. In the method according to the second aspect of the invention, the suspension prior to being concentrated can have a concentration ranging from 1 to 100×10⁶ cells/mL and preferably from 1×10⁶ to 30×10⁶ cells/mL.

The methods of the present invention are performed in order to dewater the aqueous biomass suspension for the separation of the aqueous components and the materials contained in the aqueous components from the algal cells in the aqueous suspension to provide a concentrated suspension of algal biomass. The concentrated suspension preferably has a range of from 2 to 30×10¹⁰ cells/mL and preferably from 2×10⁶ to 10×10¹⁰ cells/mL. The concentrated suspension obtained according to the method as defined in the second aspect of the invention can have a concentration ranging from 1×10⁶ to 30×10¹⁰ cells/mL and preferably from 2×10⁶ to 10×10¹⁰ cells/mL.

The method of the invention preferably produces between about 2 to 1000 and preferably from 100 to 500 times more concentrated algal biomass than the suspension prior to concentration.

The water that is separated and filtered from the algal biomass suspension can be reused for further culture of algae and as such, the method of the present invention also may be used in a bioremediation or water purification system.

The method as defined in the second aspect of the invention can further include prior to step (b) the step of recycling the concentrated suspension obtained in the ultrafiltration step until the suspension produced reaches a desired concentration. The desired concentration can range from 1×10⁶ to 30×10¹⁰ cells/mL and preferably from 2×10⁶ to 10×10¹⁰ cells/mL or can be from 2 to 1,000 and preferably from 100 to 500 times more concentrated than the suspension prior to concentration.

During the filtration process, a fresh suspension of algal biomass can be added into the reservoir that comprises the original aqueous algal biomass suspension.

The method may further comprise the step of recovering the concentrated suspension of algal biomass.

In specific aspects of the present invention the ultrafiltration method is a continuous method involving the step of passing the algal suspension through a tangential filtering device fitted with an ultrafiltration membrane.

In the methods of the invention and in the apparatus for carrying out the method described herein the tangential filtering device can comprise a cartridge containing a one or a plurality of spaced-apart parallel tubular membranes, wherein membranes have porous walls with pores of a predetermined molecular weight cut-off.

The tangential filtering device can comprise a plurality of tangential filtration cartridges arranged in fluid flow communication with one another or in parallel relationship to one another. Preferably, the tangential filtration cartridges each contain a plurality of spaced-apart parallel tubular members, wherein the tubular members have porous walls with pores of a predetermined molecular weight cut-off.

The molecular weight cut-off of the pores of ultrafiltration membranes used herein range from 1,000 to 50,000 Daltons and preferably from 5,000 to 20,000 Daltons. Preferably, the membranes are hollow fiber ultrafiltration membranes. The tubular members can define a total filtration surface ranging from 0.03 to 300 m², preferably from 5 to 130 m² and even more preferably from 10 to 25 m². In specific embodiments the membrane used is described in U.S. Pat. No. 7,435,348. This polyvinyl chloride hollow filtration membrane is one which mainly comprises 30-95 wt % PVC and 5-70 wt % vinyl-chloride-vinyl acetate-maleic anhydride terpolymer wherein the polymerization degree of PVC is from 700 to 2500, the content of vinyl acetate is 10-19% and the content of maleic anhydride is 18-40% on the basis of total weight of the terpolymer and the absolute viscosity of the terpolymer is 1.2-1.9 mPas. Preferably, the PVC hollow filtration membrane has a PVC content of 60-80 wt % and the content of the terpolymer is 20-40%. Alternatively, the PVC hollow fiber membrane has a vinyl acetate content of 13-15% and a maleic acid anhydride content of 20-28% on the basis of the total vinyl-chloride-vinyl acetate-maleic anhydride terpolymer. Method of making such a membrane are described in U.S. Pat. No. 7,435,348, incorporated herein by reference in its entirety.

In the methods of the invention, the suspension passing through the tangential filtering device can have a flow rate ranging from 1 to 5,000, preferably from 100 to 1,000 and more preferably from 250 to 500 L/hour. Still more preferably, the flux, is from 30 to 200 L/m²h and more preferably from 40 to 150 L/m²h The pressure of the suspension passing through the tangential filtering device can range from 1 to 20 psi and preferably from 3 to 10 psi. The tangential filtering device can be disposed vertically and the suspension is passed therethrough upwardly or they can be disposed horizontally.

It has been discovered herein that use of a continuous air-based backwash step, step (b) allows continuous operation of the tangential flow filtration method described herein at the desired flow rate. Preferably, the air-based backwash step is performed at least once every 10-60 minutes. Preferably, the air-based backwash is performed every 10 minutes, every 15 minutes, every 20 minutes, every 30 minutes, every 35 minutes, every 40 minutes, every 45 minutes, every 50 minutes, or every 60 minutes.

In using this backwash step it is possible to maintain an average flow rate of from about 30 L/m²h to about 100 L/m²h. Preferably, the flow rate is at least about 30 L/m²h, at least about 35 L/m²h, at least about 40 L/m²h, at least about 45 L/m²h, at least about 50 L/m²h, at least about 55 L/m²h, or at least about 60 L/m²h. In exemplary embodiments, it was found that inclusion of an air-based backwash step in the continuous cross-flow ultrafiltration method every 15 minutes resulted in a permeate flow rate of 49.69 L/m²h. This rate of about 50 L/m²h, was a dramatic increase from a rate of 35.59 L/m²h which was observed when the backwash interval was every 60 min. Further shortening the backwash interval to 10 min only resulted in a small increase in the average permeability, to 52.17 L/m²h. Based on the average permeability and operating cost, a 15 min backwash interval was chosen as optimal.

In further aspects of the invention, the method comprises an additional step of chemically declogging the membrane in order to remove the algogenic organic matter. This chemical cleaning of the membrane filters occurred when the permeability after backwashing dropped to that less than 60% of the initial permeability. Cleaning solution is pumped into the membrane module through the feed side and allowed to circulate for one hour. The cleaning solution may be any cleaning solution that can remove organic matter from a membrane filter without destroying the integrity of the underlying filter. Exemplary cleaning solutions include those that comprise about 1% to about 5% NaOH, about 0.1% to about 2% citric acid, about 50 mg/L to about 1000 mg/L NaClO, about 0.5% to about 5% EDTA and about 0.5% to about 5% surfactants. Several exemplary cleaning solutions were employed, such as 2% NaOH, 0.5% citric acid, 200 mg/L NaClO, and 400 mg/L NaClO.

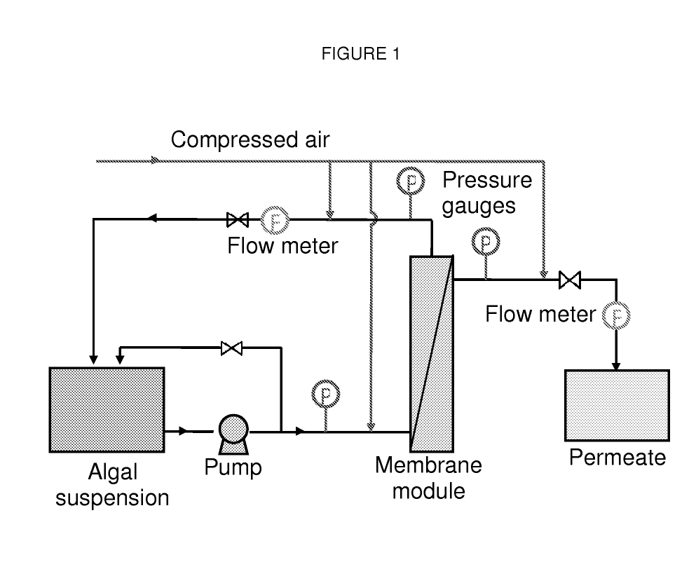

FIG. 1 shows a schematic of an apparatus that can be used to conduct the methods of the present invention. The algal suspension is in continuous flow filtration method. The suspension is filtered through the filtration apparatus such that the permeate is separated and all particles or agents larger than the 50 kD cutoff size of the membrane remain in the algal suspension. In this manner the aqueous component is continuously being removed from the algal suspension. As this continuous removal clogs the membrane filter intermittently during the continuous cross-flow filtration method, an air-backwash is performed using compressed air. This removes algal and other material that may be adhering to the membrane and causing a decrease in the flow rate of the permeate through the apparatus.

As used herein, the term “algae” or “algal strain” includes both microalgae and cyanobacteria. In one embodiment, the algae are eukaryotic microalgae. Non-limiting algal strains that can be used with the methods of the invention include but are not limited to one or more further algal strains are selected from the group consisting of Pinguiococcus pyrenoidosus, Aphanocapsa sp., Biddulphia aurita, Crypthecodinium sp., Emiliania huxleyi, Nitzschia alba, Prymnesium parvum, Skeletonema costatum, and Trichodesmium erythraeum.

The microalgae used the methods and the apparatus of the invention can be marine or freshwater microalgae. The microalgae can be selected from the group consisting of non-motile unicellular algae, flagellates, diatoms and blue-green algae. The microalgae can belong to the family of Chlorophyceae, Prasinophyceae, Bacillariophyceae, Cryptophyceae, Chrysophyceae, Haptophyceae or Cyanophyceae. The microalgae can belong to a species selected from the group consisting of Isochrysis galbana, Monochrysis lutheri, Chaetoceros muelleri and Nannochloropsis sp. The microalgae can have a size ranging from 1 to 100 μm and preferably from 3 to 20 μm.

Once the concentrated preparations of algae are prepared according to the methods described herein, the algae may be grown in any type of system or photobioreactor e.g., to be used as a source of feedstock. As used herein, a “photobioreactor” is an industrial-scale culture vessel made of transparent clear materials (e.g., glass, acrylic, polycarbonate, PVC, etc) in which algae grow and proliferate. For use in this aspect of the invention, any type of system or photobioreactor can be used, including but not limited to open raceways (i.e. shallow ponds (water level ca. 15 to 30 cm high) each covering an area of 1,000 to 5,000 m² constructed as a loop in which the culture is circulated by a paddle-wheel, closed systems, i.e. photobioreactors made of transparent tubes or containers in which the culture is mixed by either a pump or air bubbling, tubular photobioreactors and flat plate-type photobioreactors.

The algal preparations prepared and concentrated by the methods of the present invention may be used in any of a variety of methods for conversion of photosynthetic derived materials into biodiesel. Many such methods are known in the art and any such known method may be used on the algae concentrated herein. For example, the algae may be harvested, separated from the liquid medium, lysed and the oil content separated. The algal-produced oil will be rich in triglycerides. Such oils may be converted into biodiesel using well-known methods, such as the Connemann process (see, e.g., U.S. Pat. No. 5,354,878, incorporated herein by reference). Standard transesterification processes involve an alkaline catalyzed transesterification reaction between the triglyceride and an alcohol, typically methanol. The fatty acids of the triglyceride are transferred to methanol, producing alkyl esters (biodiesel) and releasing glycerol. The glycerol is removed and may be used for other purposes.

In other examples, the Connemann process (U.S. Pat. No. 5,354,878) may be used or a batch process may be used for biofuel preparation (e.g., J. Am. Oil Soc. 61:343, 1984). The Connemann process is a continuous flow of the reaction mixture through reactor columns, in which the flow rate is lower than the sinking rate of glycerin. This results in the continuous separation of glycerin from the biodiesel. The reaction mixture may be processed through further reactor columns to complete the transesterification process. Residual methanol, glycerine, free fatty acids and catalyst may be removed by aqueous extraction. The Connemann process is well-established for production of biodiesel from plant sources such as rapeseed oil and as of 2003 was used in Germany for production of about 1 million tons of biodiesel per year (Bockey, “Biodiesel production and marketing in Germany.

Other methods for producing biodiesel from triglyceride containing oils are described in e.g., U.S. Pat. Nos. 4,695,411; 5,338,471; 5,730,029; 6,538,146; 6,960,672, each incorporated herein by reference.

WO 2008/036654 provides specific exemplary methods in methods and compositions of producing medium chain fatty acid compositions from algal preparations and such methods and compositions can be performed on algal preparations prepared according to the present invention.

In certain embodiments, the concentrated algal preparations may be used for culture of animal or human-edible algae. For example, Spirulina is a planktonic blue-green algae that is rich in nutrients, such as protein, amino acids, vitamin B-12 and carotenoids. Human consumption of Spirulina grown in algal farms amounts to more than one thousand metric tons annually. The skilled artisan will realize that any type of free-living algae may be grown, harvested and utilized by the claimed system, including edible algae like Spirulina, Dunaliella or Tetraselmis (see U.S. Pat. Nos. 6,156,561 and 6,986,323, each incorporated herein by reference.)

Other algal-based products may also be produced using the aqueous algal preparations prepared according to the claimed methods. For example, U.S. Pat. No. 5,250,427, incorporated herein by reference, discloses methods for photoconversion of organic materials such as algae into biologically-degradable plastics. Any such known method for producing useful products by culture of either normal or transgenic algae may be used.

### EXAMPLE 1

### Experimental Procedures

Characterization of Algae. Scenedesmus quadricauda was obtained from our outdoor algal panel photobioreactor, and BG11 culture medium (17) was used to grow the algae. pH of the culture ranged from 7.0˜8.9 during the experimental period. Daily maximum temperature was 35° C. and minimum temperature was 15° C. Daily maximum sunlight intensity was 1900 μmol/(m²·s).The size distribution of the stain was measured by micro-flow imaging (DFA 4100, Brightwell Technologies Inc., ON, Canada). The morphology and shape of the algae were observed with a microscope.

Membrane System and Algae Concentration Process. The batch algae concentration experiment employed a lab scale polyvinylchloride (PVC) ultrafiltration (UF) membrane unit with a membrane molecular weight cutoff (MWCO) of 50 kDa and a 0.125 m² filtration area because of its excellent chemical resistance. The batch experiment was run under constant pressure. To reduce the dilution of the algae suspension by the permeate during the backwash process, instead of the backwash pump, compressed air was used to push water in the housing out of the module through the membrane. To enhance efficiency, during the backwash process pulsated air scouring was used to flush foulants on the membrane. In pulsated air scouring, the fiber is scoured with air from top to bottom for six seconds, then from bottom to top for six seconds. The VRF and concentration factor (CF) of the algae by membrane process were used for the evaluation of harvesting efficiency according to Eq (1) and Eq (2):

$$\begin{matrix}VRF = \frac{{V}_{0}}{{V}_{f}} & Eq ( 1 ) \\ CF = \frac{{C}_{f}}{{C}_{0}} & Eq ( 2 )\end{matrix}$$

where Vₒ and Cₒ are, respectively, the initial volume (m³) and the algal volumetric concentration (g/L), while V_{f} and C_{f} are the final volume (m³) and algal volume concentration.

To evaluate the harvesting efficiency, productivity (g/m²h, dry weight) was evaluated using the following equation:

$$η = \frac{3600 {C}_{f} {V}_{f}}{A \cdot t} \qquad Eq ( 3 )$$

where A is membrane filtration area (m²) and t is the concentration time (s).

Characterization of Foulants. To obtain information about the foulants adhering to the membrane, a module was autopsied before backwashing. The membrane fibers were removed and dried. To acquire images of both the inner side and the cross-section, at the end of the fiber, some top part was further removed. After coating the samples with gold, a scanning electron microscope (SEM) (Philips XL30, FEI Company, USA) equipped with an energy dispersive X-ray analysis (EDX) system was used to determine the morphology and chemical composition of the foulants on the membrane.

To identify the functional groups of the organic foulants adsorbed on the membrane, a fiber was removed from the module after backwashing, and spectra were collected using a Perkin Elmer System 2000 Fourier transform infrared (FTIR) spectrometer. The instrument scanned from 3400 to 650 cm⁻¹, averaging 10 scans at 1.0 cm⁻¹ intervals with a resolution of 4.0 cm⁻¹.

Chemical Cleaning. Fouled membranes were cleaned when the permeability after backwashing dropped to that less than 60% of the initial permeability. During the chemical cleaning process, the cross flow was closed and the dead-end configuration used. Cleaning solution was pumped into the membrane module through the feed side and allowed to circulate for one hour. Several cleaning solutions were employed, such as 2% NaOH, 0.5% citric acid and 200 mg/L NaClO, and the cleaning efficiency was evaluated by measuring the recovered flux after each cleaning step. Because the NaClO solution proved most efficient, its concentration and soaking time were further optimized.

Model Development. During the algae concentration process, cake resistance and backwash irreversible resistance due to adsorption on the filter resulted in a flux decline. The thickness of the cake layer increased with time until it reached a steady state at which the rate of algae deposition onto the membrane equaled the rate of algae transport from the membrane to the bulk solution. The flux achieved a corresponding steady state (18). At certain intervals, backwashing was used to remove cake buildup on the surface of the membrane. The backwash process cannot remove backwash irreversible foulants, however, so the flux decreases gradually.

During algal harvesting process algae concentration keeps increasing, resulting in the accelerated cake build up and flux drop. Nevertheless, the flux decline can be predicted if the cake resistance and backwash irreversible resistance at different times can be calculated. To develop the model, the following assumptions were made:

1) The fouling of the membrane is due to the algal cake buildup and the adsorption of AOM on the membrane. The cake buildup causes the most resistance.

2) Each backwash process removes all of the cake buildup. Backwashing cannot remove backwash irreversible fouling, so it accumulates gradually.

3) Specific algal cake resistance and backwash efficiency do not change during the concentration process..

Resistance-in-series Model. The resistance-in-series model was used to calculate the permeate flow rate. According to this model, membrane filtration can be described by Darcy's law, as shown in the following equation:

$$Q = \frac{A Δ P}{μ ( {R}_{m} + {R}_{c} + {R}_{ir} )} \qquad Eq ( 4 )$$

where Q (m³/s) is the permeation flow rate, ΔP is the TMP (transmembrane pressure), is the viscosity of the permeate, Rₘ is the inherent membrane resistance, R_{c} is the cake resistance, and Rᵢᵣ is the fouling resistance due to backwash irreversible adsorption.

The permeation flow rate through a membrane at any time can be calculated if the three resistances are known. Rₘ can be calculated using pure water. The calculation of R_{c} and Rᵢᵣ over time is described below.

Calculation of Backwash Irreversible Resistance. Backwash efficiency can calculated using the following equation:

$$r = \frac{{Q}_{n}}{{Q}_{n - 1}} \qquad Eq ( 5 )$$

where r is backwash efficiency, and Oₙ₋₁ and Oₙ are the flow rates after the n-1 and n backwash.

The flow rates after the n-1 and n backwashes can be calculated using using a combination of Eq (1) and Eq (5); the new equation can be written:

$${R}_{irm} = \frac{1 - r}{r } {R}_{m} + \frac{1}{r} {R}_{ir ( n - 1 )} \qquad Eq ( 6 )$$

where Rᵢᵣ₍ₙ₋₁₎ and R_{ir n} are the backwash irreversible fouling resistances after the n-1 and n backwashes. At the beginning of the filtration, Rir₀=0, and r can be calculated via the backwash experiment. Thus, Eq (6) can be used to calculate the backwash irreversible fouling.

Calculation of Cake Resistance. Calculate flux at steady state using force balance model. In the cross-flow membrane concentration process, negative direction forces such as permeation drag (F_{d}) move the algae toward the membrane surface, while positive forces such as Brownian diffusion (F_{B}), shear induced diffusion (Fₛ), and lateral inertial lift (Fₗ) shift algae away from the membrane surface (19, 20). The net force exerted on an algal particle, F, is the sum of all forces listed above (21). At a steady state the flux (Js) can be calculated using the following equation, (21):

$$\begin{matrix}Js = {v}_{B} + {v}_{s} + {v}_{l} & Eq ( 7 ) \\ {v}_{B} = \frac{0.807 {D}_{B}^{2 / 3} {τ}_{W}^{1 / 3}}{{L}^{1 / 3}} ln ( \frac{{C}_{w}}{{C}_{b}} ) & Eq ( 8 ) \\ {v}_{s} = \frac{0.807 {D}_{s}^{2 / 3} {τ}_{W}^{1 / 3}}{{L}^{1 / 3}} ln ( \frac{{C}_{w}}{{C}_{b}} ) & Eq ( 9 ) \\ {v}_{I} = 0.577 \frac{{d}_{P}^{3} {U}_{m}^{2}}{{I}^{2} v} & Eq ( 10 )\end{matrix}$$

where D_{B} is the Brownian diffusion coefficient, (D_{B}=k_{B}T/6πμd_{P}²); Dₛ is the shear-induced diffusion coefficient (Dₛ=0.03d_{P}²τ_{W}); τ_{W} is the wall shear stress (s⁻¹); C_{w} volume concentration of particles at the membrane surface; C_{b} is the volume concentration of algae in bulk solution; L is the membrane module channel length (m); l is the channel height (m); v is the kinematic viscosity (m²/s); Uₘ is the maximum flow velocity at the fiber (m/s); d_{P} is the equivalent volume radius of the algae (m); and τ is the shear rate (s⁻¹).

One important parameter is algae radius. Scenedesmus is ellipsoid, so the equivalent volume radius of a ball of the same volume as Scenedesmus was used. The equivalent volume radius was calculated using the following equation:

$${d}_{p} = \sqrt[1 / 3]{\frac{3}{4 π} ( \frac{4 π}{3} ) \cdot {( \frac{a}{2} )}^{2} \cdot \frac{b}{2}} \qquad Eq ( 11 )$$

where a and b are the diameters of the algae along the shorter and longer axes, respectively. According to the SEM results, a is about 0.39 times the length of b. b can be obtained from the equivalent circular diameter measured by MFI (4.92±0.95 μm) using the following equation:

$$\begin{matrix}b = \sqrt{\frac{{d}_{ecd}^{2}}{0.39}} & Eq ( 12 ) \\ \frac{1}{{d}_{ecd}} = \sum \frac{{p}_{i}}{{d}_{pi}} & Eq ( 13 )\end{matrix}$$

where d_{ecd} is equivalent circular diameter measured by MFI, and pᵢ is the percentage of particle dₚᵢ in the total particles.

Calculation of cake thickness and cake resistance. The cake layer is usually an immobile layer of retained particles packed at maximum density on the membrane surface. The resistance of the cake layer, R_{c}, is given as:

R_{c}=k_{c}·δ_{c} Eq (14)

where k_{c} (1/m²) is the specific resistance per unit of cake thickness, and δ_{c} (m) is the cake thickness. The value of k_{c} can be calculated from the experimental data using the following equation (22):

$$\frac{t}{{V}_{t}} = \frac{μ {k}_{c} {c}_{b}}{2 {A}^{2} Δ P} {V}_{t} + \frac{μ {R}_{m}}{A Δ P} \qquad Eq ( 15 )$$

where t is filtration time (s), and V is the permeate volume at time t (m³).

In cross-flow membrane filtration the prediction/determination of δ_{c} is problematic, as it can be affected by the operation parameters and is usually difficult to measure experimentally. However, Bai et al. (23) has developed a model to predict δ_{c}. In this model, the backwash irreversible fouling Rᵢᵣ is also taken into consideration. The calculation of δ_{c} is described below.

The cake thickness δ_{c}, in principle, can be described by the following equation (23):

$$\frac{d {δ}_{c}}{d t} = {k}_{cr} ( \frac{J}{{J}_{s}} - 1 ) \qquad Eq ( 16 )$$

where Jₛ is the flux at steady state, and K_{cr} is the rate parameter of cake growth (m/s), which can be calculated using the following equation:

$${K}_{cr} = \frac{{J}_{s}}{{J}_{0} - {J}_{s}} \cdot \frac{{C}_{b}}{{C}_{w}} \cdot {J}_{0} \qquad Eq ( 17 )$$

where C_{b} is volume concentration of algae in the bulk solution, and C_{w} is the volume concentration of particles at the membrane surface (0.925 was selected in this study based on the best fitting results). By introducing Eq (4) and Eq (14) into Eq (16), the following rate expression for cross-flow filtration can be obtained:

$$\frac{d {δ}_{c}}{d t} = \frac{{k}_{cr}}{{J}_{s}} \cdot \frac{Δ P - {J}_{s} μ ( {R}_{m} + {k}_{c} {δ}_{c} + {R}_{ir} )}{μ ( {R}_{m} + {K}_{c} {δ}_{c} + {R}_{ir} )} \qquad Eq ( 18 )$$

Integration of the equation gives the following, which can be used to calculate cake thickness by iteration:

$$- \frac{Δ P}{{J}_{s} μ {k}_{c} {k}_{cr}} ln ( 1 - \frac{{J}_{s} μ {k}_{c} {δ}_{c}}{Δ P - {J}_{s} μ ( {R}_{m} + {R}_{ir} )} ) - \frac{{δ}_{c}}{{k}_{cr }} = t \qquad Eq ( 19 )$$

In deriving Eq (18), k_{c} and k_{cr} are assumed not to change with time. If k_{c}, k_{cr} and Jₛ are known, Eq (19) can be used to determine the cake thickness at any time in a cross-flow microfiltration.

Calculation of Permeate Volume and Final Algae Concentration. The following equation was used to calculate the total volume of permeate:

$${V}_{T ( i , j )} = {\sum }_{i = 1}^{n} {\sum }_{j = 0}^{{T}_{BW} - 1} \frac{1}{2} ( {Q}_{i , j} + {Q}_{i , j + 1} ) \cdot {t}_{interval} \qquad Eq ( 20 )$$

where i is the number of the filtration cycle; j is the number of calculated steps at each cycle; n is the total number of cycles in the concentration process; T_{BW} is the backwash interval; V_{T(i,j)} is the total volume of permeate at step j in the filtration cycle of i (m³); and tᵢₙₜₑᵣᵥₐₗ is the interval between the calculation steps, 60 s (1 min) in this case.

Based on the mass balance, the algal concentration can be calculated from the following equation:

$${C}_{( i , j )} = \frac{{C}_{0} {V}_{0}}{{V}_{T ( i , j )}} \qquad Eq ( 21 )$$

where C_{(i,j)} is the algal volume concentration at step j in the filtration cycle of i; C₀ is the initial algal concentration; and V₀ is the initial volume of the algal suspension.

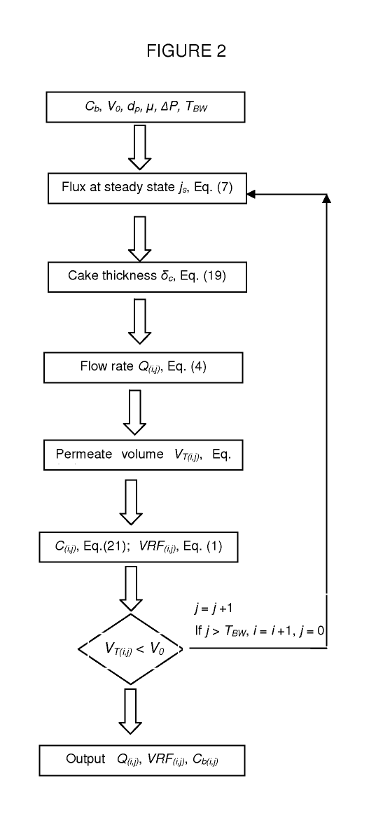

FIG. 2 presents the calculation process using the developed model. Key parameters used in the model and their values are listed in Table 1.

### EXAMPLE 2

### Results

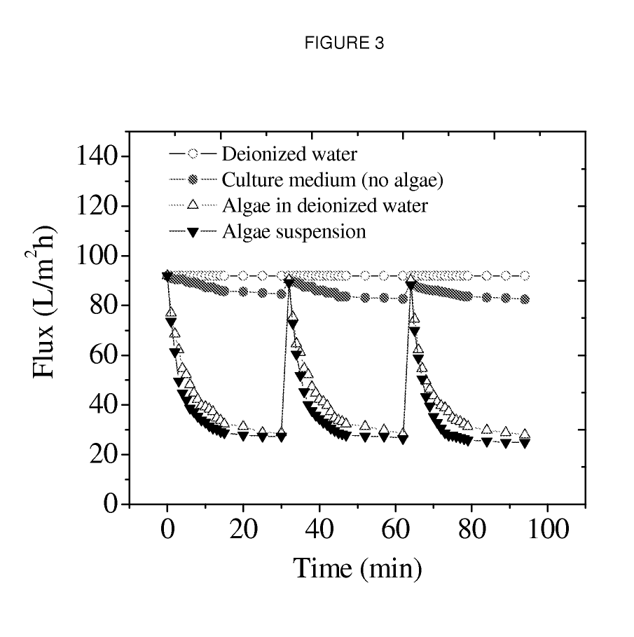

Fouling Tendency of Algae on the Membrane. FIG. 3 presents the flux decline curves for the filtration of different types of water. The flow rate was very stable when tap water was used. A slight flux rate decline was observed in the filtration of the algal culture media, indicating that it contained some foulants. Similar fast flow drops were found in the filtration of the original algal suspension (algae and culture media) and of the algal suspension in DI water, though the decline for the original suspension was slightly higher. The results indicate that the cake layer dominates the total resistance during the entire experiment. This is quite reasonable when the algal size is compared with the membrane pore size. Similar results were obtained from research focusing on the removal of Chlorella sp. and cyanobacterial cells via membrane filtration (13, 17).

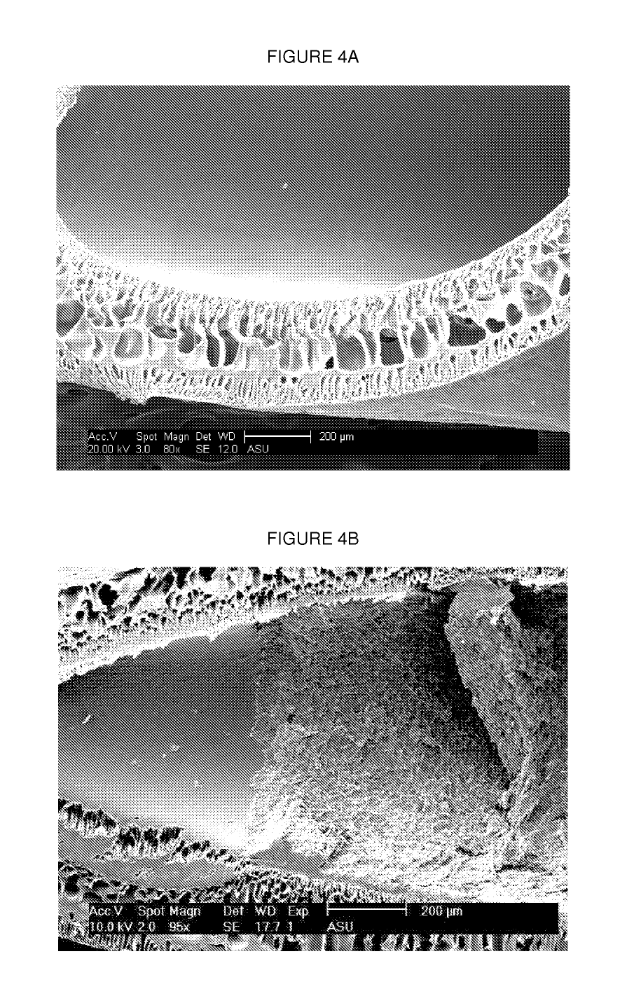

Characterization of Foulants. FIG. 4A and FIG. 4B present SEM images of virgin and fouled membranes. The inside of the virgin membrane is very smooth and clean. In contrast, a caked-on layer was observed on the inner surface of the hollow fiber of the fouled membrane. Residual foulant material was analyzed by energy dispersive x-ray analysis (EDX), and Na, Ca, Mg, and Fe were found in the cake layer.

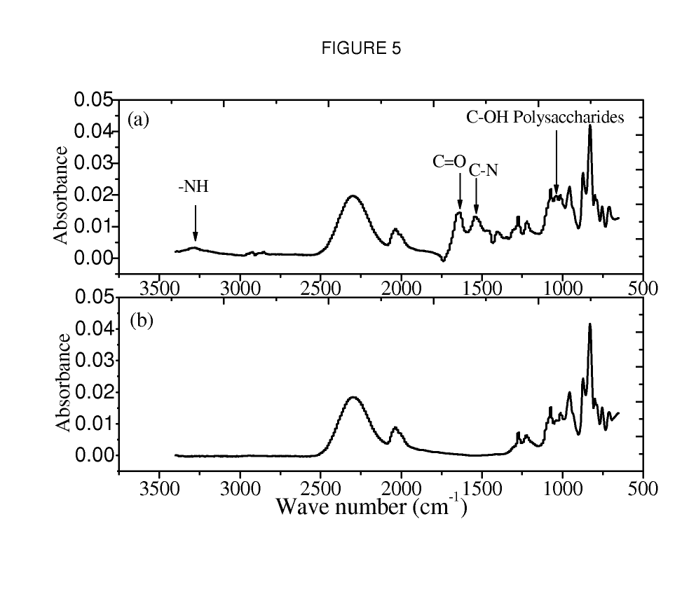

The backwash irreversible foulants were identified using FTIR. FIG. 5 presents the spectrum, which exhibits a broad region of absorption at 3300, 1640 and 1550 cm⁻¹. These are the characteristic bands for proteins, due to stretching of N—H bonds, stretching of C═O bonds (amide|band), and deformation of N—H bonds (amid∥band). Based on the peak at 1,400 cm⁻¹ (others 1,380), the membrane foulants contained some amount of lipids (24). The broad peak at 1,100 cm⁻¹ is due to C—O bonds and is associated with polysaccharides or polysaccharide-like substances (15). Lee et al. have shown that polysaccharide-like substances (amino sugars), polysaccharide groups, and proteins play a significant role in membrane fouling. The results suggest that the algal culture media contains proteins, polysaccharides and lipids, and the presence of this AOM causes backwash irreversible membrane fouling.

Optimization of the Algae Concentration Process. Based on the above analysis, most of the foulants are deposited algal cake, and the rest are adsorbed or gelled organic and biological compounds. Periodic backwashing was found to successfully remove most of the backwash reversible fouling due to cake formation (25). The adsorbed or gelled organic and biological compounds require chemical cleaning, however. The fouling control strategies for the algae concentration process are to optimize the backwash interval, cross flow velocity, and chemical cleaning process.

Five backwash intervals (5, 10, 15 30 and 60 minutes) were tested. For all tested intervals, the flux declined until the next backwash process. Shorter backwash intervals led to higher permeate flow rates and higher initial flux for each filtration cycle, indicating that frequent backwashing does help control fouling. However, frequent backwashing also decreases online working time and thus lowers the concentration efficiency. This anti-fouling operation also obviously affects operating costs, as energy is required to achieve a pressure suitable for flow reversion. Moreover, the backwash process requires additional permeate, thus diluting the algal suspension. The average flow rate was used to evaluate the backwashing performance based on 60 min's concentration. The average flow rate increased dramatically from 35.59 L/m²h to 49.69 L/m²h when the backwash interval decreased from 60 min to 15 min. Further shortening the backwash interval to 10 min only resulted in a small increase in the average permeability, to 52.17 L/m²h. Based on the average permeability and operating cost, a 15 min backwash interval was chosen as optimal.

The cross-flow velocity of the feed stream has a positive effect on the water flux. At the highest cross-flow velocity tested (0.17 m/s), a slower flux decline was observed, and the final flux at 60 min is higher (25.44 L/m²h) than those at the smaller cross flow velocities. The average permeate flow rate at 0.01, 0.09 and 0.17 m/s was 19.43, 28.03 and 39.99 L/m²h, respectively. It is as expected as the higher the cross-flow the more the deposition is limited on the membrane surface (26).

Several chemicals, including 2% NaOH, 0.5% citric acid and 200 mg/L NaClO, were used as cleaning agents, and the results showed that NaClO was most effective. Higher NaClO concentrations resulted in better cleaning. The best flux recovery occurred when 400 mg/L NaClO was used; the specific permeability increased to 90.7 L/m²h, which is 98% of the average initial (pre-harvesting) specific permeability (92.2 L/m²h) of the membrane. Further increases in NaClO concentration did not increase the specific permeability. NaClO is the chemical most extensively used for membrane disinfection and biofouling control (21). The action of NaClO as a swelling agent and protein solubilizer, in combination with its ability to break the chemical bonds between foulants and the membrane, makes it very effective (27). Li also showed the effectiveness of NaClO in cleaning membranes used for the treatment of algae rich water (28).

### EXAMPLE 3

### Model Validation

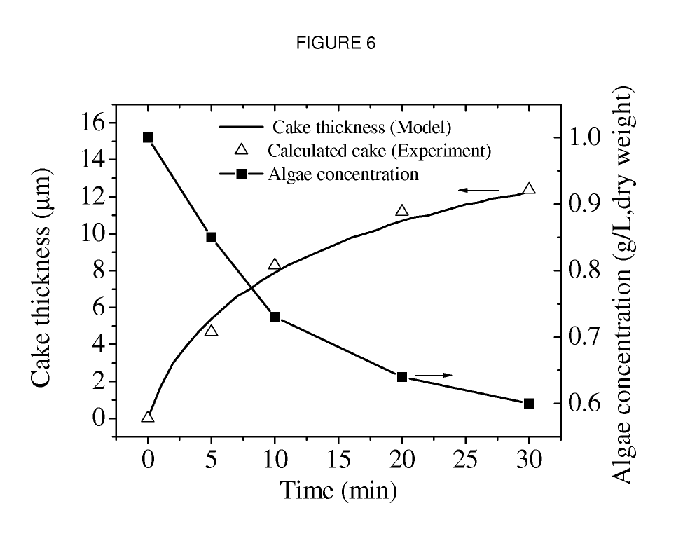

Modeling of cake thickness development. During the concentration process, the permeate was returned to the feed tank, and the algal concentration was tested at different times. Based on the mass balance, the thickness of the algal cake layer was calculated. FIG. 6 shows the decrease in algal concentration and the increase in cake thickness. The thickness of the algal cake layer also was calculated using the model. As shown in the figure, the algal concentration in the feed tank decreased gradually due to the cake attached to the membrane surface. The deposited cake layer was about 12.3 microns thick after 30 min filtration. The cake thickness predicted by the model agrees with the experimental data (R²=0.993), indicating that the model works well.

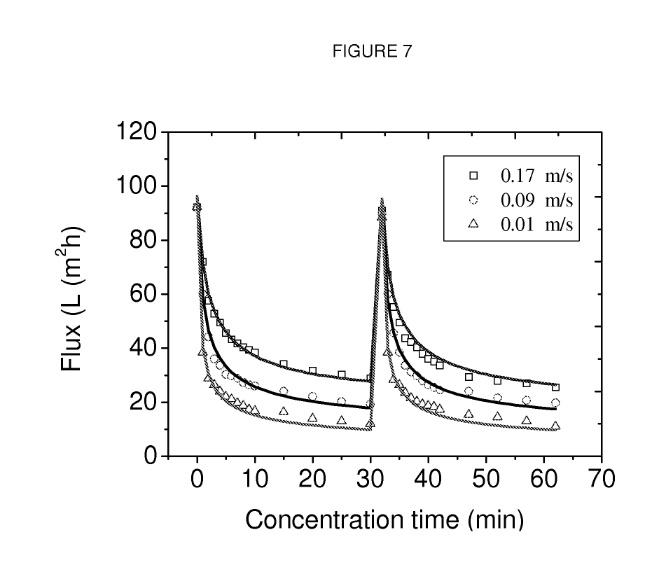

Modeling of the flux decline under different cross flow rates. Further validation of the model was conducted by predicting the flux decline under different conditions. FIG. 7 shows the experimental and model-predicted flux decline under different cross flow rates. As discussed in the above section, higher shear velocity makes it harder for algae to deposit on the membrane, thus induces higher flux. The data predicted by the model are very similar to the experimental data obtained (R²>0.991).

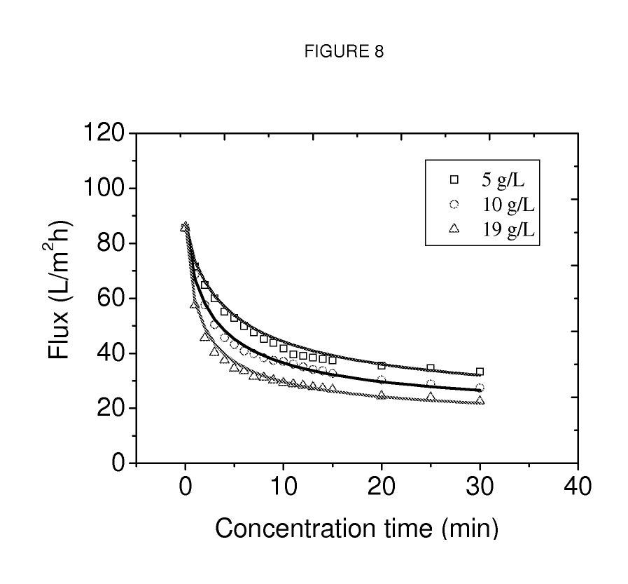

Modeling of the flux decline under different initial algal concentrations. The model also predicted the influence of the initial algal concentration; FIG. 8 presents both experimental and modeled results. For higher algal concentrations, algal cake builds up faster, and thus the flux declines faster. In addition, for higher initial algal concentrations, the steady state flux is lower. The model predictions are consistent with the experimental results (R²>0.990).

### EXAMPLE 4

### Evaluation of a Long Term Concentration Cycle and Modeling of the Concentration Process

A long term algal concentration experiment was conducted to test the extended performance of the UF membrane and evaluate the model. No algae were found in the permeate, which had an average turbidity of 0.018 NTU.

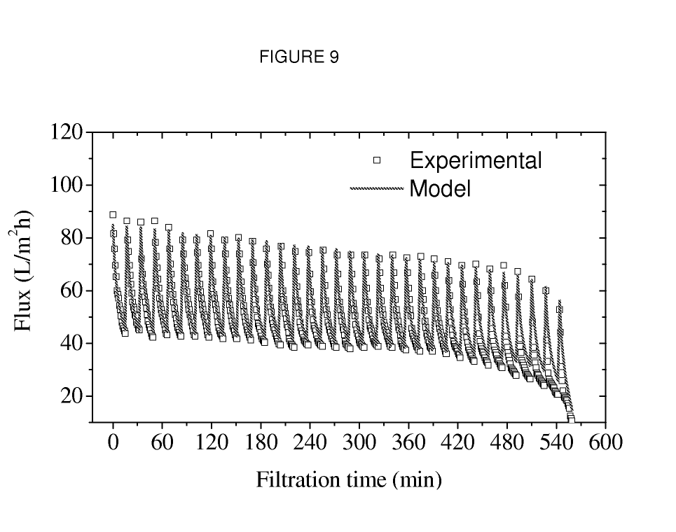

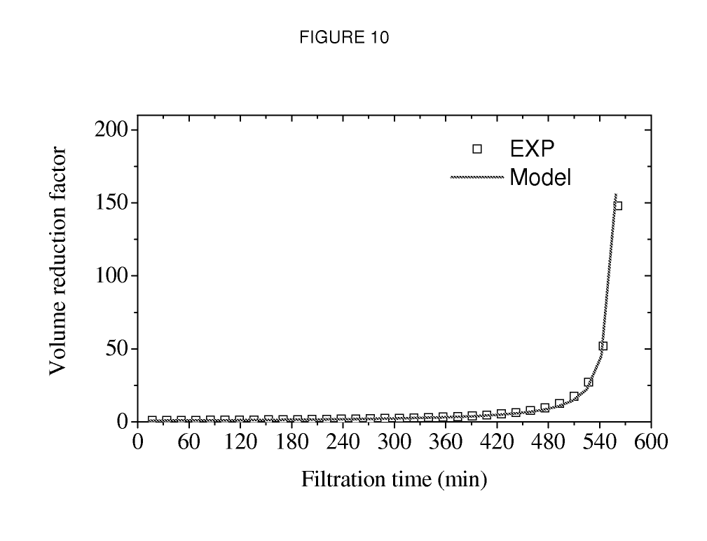

FIG. 9 presents profiles of the flux decline for both experimental and model values. The flux declined in each cycle, but the backwash process was sufficiently efficient to restore the flux. Much faster flux declines were observed when the algal concentration was higher. Due to the backwash irreversible fouling, not all of the permeate flux is restored after each backwash process. Thus, as can be seen in FIG. 10, the initial flux decreased with each cycle. A significant decrease was observed during the final stage of the concentration process, when the algal concentration rose. A comparison of the theoretical simulations and experimental measurements shows that the model correctly predicts the flux decline at different initial algal concentrations.

FIG. 11 presents the changes in the VR with time for both experimental and modeled data. VRF increased with time as permeate was extracted from the algal suspension. A rapid increase in VRF was obtained when the algal concentration decreased. The VRF at 559 min was approximately 150 and 155 in the experiment and the model, respectively.

In our experiment final CF is 154, and algae was concentrated to 170 g/L. The calculated algae harvesting capacity is 46.01 g/m²h (dry weight). Considering water reclamation aspects, the average permeability is 45.50 L/m²h at an initial algal concentration of 1.04 g/L. A comparison of VRF under different experimental conditions was presented in Table 2. A much higher VRF (150) was obtained in our experiment, which is due to the adoption of different anti-fouling strategies in the concentration process. The results show that the concentration of the algal suspension using UF membrane is very promising under the optimized conditions described.

### Nomenclature:

A membrane filtration area (m²)
a diameter of the algae along the longer axis (m)
b diameter of the algae along the shorter axis (m)
C₀ initial algal concentration
C_{b} volume concentration of algae in the bulk solution (%)
C_{(i,j)} algal volume concentration at step j in filtration cycle i
C_{f} final algal concentration
CF concentration factor
C_{w} volume concentration of algae at the membrane surface (%)
D_{B} Brownian diffusion coefficient
d_{ecd} equivalent circular diameter of the algae (m)
dₚ equivalent volume radius (m)
d_{Pi} range of algal radius (m)
Ds shear-induced diffusion coefficient
F_{d} permeation drag force that moves the algae toward the membrane surface
F_{B} Brownian diffusion force
Fₛ shear induced diffusion
Fᵢ lateral inertial lift force
l channel height (m)
i number of the filtration cycle
j number of the calculated step at each cycle
J₀ flux of the membrane before the initial filtration cycle (m³/(m²·s))
Jₛ permeation flux at steady state (m³/m²s)
k_{c} specific cake resistance (m⁻²)
k_{cr} cake growth rate (m/s)
k_{B} Boltzmann constant
L membrane module channel length (m)
n number of total cycles in the concentration process
pᵢ percentage of particles of dₚᵢ in the total particles
Q permeate flow rate (m³/s)
Qₙ₋₁ flow rate after the n-1 backwash (m³/s)
Qₙ flow rate after the n backwash (m³/s)
r flux recovery after backwashing
R_{c} cake resistance (m⁻¹)
Rₘ inherent membrane resistance
Rᵢᵣ backwash irreversible resistance due to strong attachment, adsorption or chemical bonding
Rᵢᵣ₍ₙ₋₁₎ backwash irreversible fouling resistance after the n-1 backwash
R_{ir n} backwash irreversible fouling resistance after the n backwash
Uₘ cross flow velocity (m/s)
V₀ initial volume of the algal suspension (L)
v_{B} algal transport velocity due to Brownian diffusion (m/s)
vᵢ algal transport velocity due to lateral inertial lift (m/s)
Vₜ final volume (L)
VRF volumetric reduction factor
vₛ algal transport velocity due to shear induced diffusion (m/s)
V_{T(i,j)} total volume of the permeate at step j in filtration cycle i
tᵢₙₜₑᵣᵥₐₗ interval between the calculation steps
T temperature, Kelvin
T_{BW} backwash interval

Greek Symbols
δ cake thickness (m)
ΔP transmembrane pressure (Pa)
μ dynamic viscosity (Pa·s)
v kinematic viscosity (m²/s)
η algal concentration productivity (g/(m²·h), dry weight)
T_{W} wall shear stress (s⁻¹)

TABLE 1
Key parameters used in the model and their values

| Parameters | Unit | Value |
|---|---|---|
| Algae equivalent volume radius, dₚ | m | 1.7 × 10⁻⁶ |
| Initial algae bulk concentration, C_{b} | % | 0.104 |
| Initial volume of the algal suspension, V₀ | L | 53.4 |
| TMP, ΔP | Pa | 31026 |
| Cross flow velocity, Uₘ | m/s | 0.17 |
| Specific cake resistance k_{c} | 1/m² | 9.09 × 10¹⁶ |
| Cake growth rate k_{cr} | m/s | 9.17 × 10⁻⁹ |
| Algal volume concentration at membrane | % | 92.5 |
| surface C_{w} | | |

TABLE 2
Comparison of VRF

| Membrane | Algal species | Initial C (g/L) | VRF | Ref |
|---|---|---|---|---|
| Millipore, | Scenedesmus, | chlorophyll-α | 5 to 40 | (11) |
| 0.45 μm | Monoraphidium sp., | <2.5 μg/L | | |
| | Navicula sp et al. | | | |
| | Cyanobacterium, | | | |
| Polyacrylonitrile | Arthrospira platensis | 0.45 | 10 | (29) |
| (40 kDa) | | | | |
| PVC UF | Scenedesmus, | 1.0 | 150 | This |
| (50 KDa) | | | | study |

### REFERENCES CITED

1. Wang B, L. Y., Wu N, Lan CQ ., CO2 bio-mitigation using microalga. Appl Microbiol Biotechnol., 2008. 79(5): p. 707-718.

2. Gudin, C. and C. Thepenier, Bioconversion of Solar Energy into Organic Chemicals by Microalgae. Mizrahi, a. (Ed.). Advances in Biotechnological Processes, Vol. 6. Xvii 296p. Alan R. Liss, Inc.: New York, N.Y., USA. Illus, 1986: p. 73-110.

3. Munoz, R. and B. Guieysse, Algal-bacterial processes for the treatment of hazardous contaminants: A review. Water Research, 2006. 40(15): p. 2799-2815.

4. Heasman, M., et al., Development of extended shelf-life microalgae concentrate diets harvested by centrifugation for bivalve molluscs—a summary. Aquaculture Research, 2000. 31(8-9): p. 637-659.

5. Carmichael, W. W., C. Drapeau, and D. M. Anderson, Harvesting of Aphanizomenon flos-aquae Ralfs ex Born. & Flah. var. flos-aquae (Cyanobacteria) from Klamath Lake for human dietary use. Journal of Applied Phycology, 2000. 12(6): p. 585-595.

6. Nurdogan, Y. and W. J. Oswald, Tube settling of high-rate pond algae. Water Science and Technology, 1996. 33(7): p. 229-241.

7. Yahi, H., S. Elmaleh, and J. Coma, Algal Flocculation-Sedimentation by Ph Increase in a Continuous Reactor. Water Science and Technology, 1994. 30(8): p. 259-267.

8. Elmaleh, S., et al., Magnesium Induced Algal Flocculation in a Fluidized-Bed. Water Science and Technology, 1991. 23(7-9): p. 1695-1702.

9. Sim, T. S., A. Goh, and E. W. Becker, Comparison of Centrifugation, Dissolved Air Flotation and Drum Filtration Techniques for Harvesting Sewage-Grown Algae. Biomass, 1988. 16(1): p. 51-62.

10. Rossignol, N., et al., Membrane technology for the continuous separation microalgae/culture medium: compared performances of cross-flow microfiltration and ultra-filtration. Aquacultural Engineering, 1999. 20(3): p. 191-208.

11. Petrusevski, B., et al., Tangential flow filtration: A method to concentrate freshwater algae. Water Research, 1995. 29(5): p. 1419-1424.

12. Heng, L., et al., Effect of pretreatment by permanganate/chlorine on algae fouling control for ultrafiltration (UF) membrane system. Desalination, 2008. 222(1-3): p. 74-80.

13. Hung, M. T. and J. C. Liu, Microfiltration for separation of green algae from water. Colloids and Surfaces B: Biointerfaces, 2006. 51(2): p. 157-164.

14. Kwon, B., N. Park, and J. Cho, Effect of algae on fouling and efficiency of UF membranes. Desalination, 2005. 179(1-3): p. 203-214.

15. Her, N., et al., Characterizing algogenic organic matter (AOM) and evaluating associated NF membrane fouling. Water Research, 2004. 38(6): p. 1427-1438.

16. Babel, S., S. Takizawa, and H. Ozaki, Factors affecting seasonal variation of membrane filtration resistance caused by Chlorella algae. Water Research, 2002. 36(5): p. 1193-1202.

17. Andersen, R. A., ed. Algal Culturing Techniques. 2005, Academic Press.

18. Field, R. W., et al., Critical flux concept for microfiltration fouling. Journal of Membrane Science, 1995. 100(3): p. 259-272.

19. Altena, F.W. and G. Belfort, Lateral migration of spherical particles in porous flow channels: application to membrane filtration. Chemical Engineering Science, 1984. 39(2): p. 343-355.

20. Belfort, G., Fluid mechanics in membrane filtration: Recent developments. Journal of Membrane Science, 1989. 40(2): p. 123-147.

21. Lee, J.-D., et al., Effect of Coagulation Conditions on Membrane Filtration Characteristics in Coagulation &#x2212; Microfiltration Process for Water Treatment. Environmental Science & Technology, 2000. 34(17): p. 3780-3788.

22. A. Rushton, A. S. W., R. G. Holdich, Filtration Fundamentals, in Solid-Liquid Filtration and Separation Technology. 1996, VCH: Weinheim; New York; Basel; Cambridge; Tokyo p. I-XIII.

23. Bai, R. and H. F. Leow, Microfiltration of activated sludge wastewater—the effect of system operation parameters. Separation and Purification Technology, 2002. 29(2): p. 189-198.

24. Ramesh, A., D. J. Lee, and J. Y. Lai, Membrane biofouling by extracellular polymeric substances or soluble microbial products from membrane bioreactor sludge. Applied Microbiology and Biotechnology, 2007. 74(3): p. 699-707.

25. C. Gourgues, P. Aimar, V. Sanchez, Ultrafiltration of bentonite suspensions with hollow fiber membranes J. Memb. Sci. 74 (1992) 51˜69.

26. Belfort, G., R. H. Davis, and A. L. Zydney, The behavior of suspensions and macromolecular solutions in crossflow microfiltration. Journal of Membrane Science, 1994. 96(1-2): p. 1-58.

27. Daufin, G., et al., Cleaning of inorganic membranes after whey and milk ultrafiltration. Biotechnology and Bioengineering, 1991. 38(1): p. 82-89.

28. Liang, H., et al., Cleaning of fouled ultrafiltration (UF) membrane by algae during reservoir water treatment. Desalination, 2008. 220(1-3): p. 267-272.

29. Rossi, N., et al., Harvesting of Cyanobacterium Arthrospira Platensis Using Organic Filtration Membranes. Food and Bioproducts Processing, 2004. 82(3): p. 244-250.

1. A method of dewatering an aqueous algal biomass comprising:
a. subjecting an initial aqueous algal biomass to a continuous cross-flow ultrafiltration to remove water from said aqueous algal biomass, wherein said ultrafiltration is performed using a hollow-fiber PVC ultrafiltration membrane with a molecular weight cutoff off of 50 kDa; and
b. intermittently subjecting said ultrafiltration membrane to an air-based backwash to remove algae from said membrane
wherein said method produces at least a 50-fold increase in algal biomass in said aqueous suspension as compared to the algal biomass in the starting aqueous suspension, wherein said method optionally further comprises collecting the water removed from said algal biomass.

2. (canceled)

3. The method of claim 1, wherein said initial aqueous biomass comprises an algal biomass of 1.04 g/L cell dry weight, and said continuous ultrafiltration produces an aqueous algal biomass suspension comprising about 50 to 200 g/L cell dry weight.

4. The method of claim 1, wherein said air-based backwash is performed at a time interval of between 10 to 60 minutes.

5. The method of claim 1, wherein said air-based backwash is performed at a time interval selected from the group consisting of every 10 minutes, every 15 minutes, every 20 minutes, every 25 minutes, every 30 minutes, every 35 minutes, every 40 minutes, every 45 minutes, every 50 minutes, every 55 minutes and every 60 minutes.

6. The method of claim 1 further comprising one or more of: a) monitoring the average flow rate of the permeate through the membrane; and b) declogging said membrane to remove algogenic organic matter therefrom.

7. (canceled)

8. The method of claim 6, wherein said membrane is declogged when the flux of the permeate through said membrane decreases to a value between 20 to 70 L/m²h.

9. The method of claim 6, wherein said membrane is declogged when the flow rate of the permeate through said membrane decreases to less than 50% of the flow rate of the permeate through new membrane.

10. The method of claim 6, wherein said membrane is declogged by chemical cleaning when the flow rate of the permeate after backwashing dropped to that less than 60% of the initial flow rate of the permeate through the membrane.

11. The method of claim 6, wherein said declogging comprises a soaking step wherein said cross-flow ultrafiltration is stopped and the ultrafiltration membrane is soaked with a circulating fluid comprising a cleaning solution selected from the group consisting of about 1% to about 5% NaOH, about 0.1% to about 2% citric acid, about 50 mg/L to about 1000 mg/L NaClO, about 0.5% to about 5% EDTA and about 0.5% to about 5% surfactants.

12. The method of claim 6, wherein said cleaning solution comprises about 400 mg/L NaClO.

13. The method of claim 1, wherein said algal biomass is contained in a water sample that is a freshwater sample or a marine water sample.

14. (canceled)

15. The method of claim 1, wherein said algal biomass comprises one or more algae belonging to a family of selected from the group consisting of Chlorophyceae, Prasinophyceae, Bacillariophyceae, Cryptophyceae, Chrysophyceae, Haptophyceae and Cyanophyceae.

16. The method of claim 1, wherein said starting aqueous suspension comprises between 1 to 100×10⁶ cells/mL.

17. The method of claim 15, wherein said method concentrates the algal cells in said suspension to a concentration of between about 1×10⁶ to 30×10¹⁰ cells/mL.

18. An isolated aqueous suspension of algae isolated according to the method claim 1, wherein at least 90% of the algal cells in said population are live, unruptured algal cells.

19. A method of recycling an aqueous solution containing an algal biomass comprising the steps of:
a. subjecting an initial aqueous solution containing algae and other microorganisms to a continuous cross-flow ultrafiltration to remove water from said aqueous algal biomass, wherein said ultrafiltration is performed using a hollow-fiber PVC ultrafiltration membrane with a molecular weight cutoff off of 50 kDa;
b. intermittently subjecting said ultrafiltration membrane to an air-based backwash to remove algae from said membrane; and
c. collecting the permeate isolated from said ultrafiltration step.

20-22. (canceled)

23. A cross-flow membrane filtration method for the removal or separation of algal cells from an aqueous environment comprising:
a. subjecting an initial aqueous algal biomass to a continuous cross-ultrafiltration to remove water from said aqueous algal biomass, wherein said ultrafiltration is performed using a hollow-fiber PVC ultrafiltration membrane with a molecular weight cutoff off of 50 kDa; and
b. subjecting said ultrafiltration membrane to an air-based backwash to remove algae from said membrane at least once every 30 minutes; and
c. chemically declogging said ultrafiltration membrane when the average flow rate of the permeate after backwashing dropped to less than 60% of the initial average flow rate of the permeate through the membrane;
wherein said method produces at least a 50-fold increase in algal biomass in said aqueous suspension as compared to the algal biomass in the starting aqueous suspension.

24. An improved method to enhance algal biomass concentration/collection/separation/harvesting efficiency the improvement comprising using filtration by employing air scour for fouling control wherein no backwash pump is needed.

25. (canceled)

26. In method of claim 24, no water was pumped into the module to backwash the fouled membrane.

27. In method of claim 24, dilution of algae suspension due to backwash was minimized.

28. In method of claim 24, algae can be collected by scouring the membrane using air.
